(19) United States
(12) Patent Application Publication
(10) Pub. No.: US 2003/0049333 A1
(43) Pub. Date: Mar. 13, 2003

(54) Composition that comprises ozonised oils and/or other natural and/or synthetic ozonised products, and use thereof in pharmaceutical, cosmetic, dietary or food supplement products, in the human and veterinary fields

(76) Inventors: Manuel-Antonia Gomez Moraleda, Vilassar De Dalt (ES); Roberto Dall' aglio, Vilasssar De Dalt (ES); Pierangelo Melegari, Vilassar De Dalt (ES)

Correspondence Address: Joshua King, Suite. 350, 155-108th Avenue N.E., Bellevue, WA, 98004-5973, US

(21) Appl. No.: 10/155,472

(22) Filed: May 24, 2002

Related U.S. Application Data
(63) Continuation-in-part of application No. PCT/ES00/00208, filed on Jun. 9, 2000.

(30) Foreign Application Priority Data
Nov. 25, 1999 (ES) 9,902,602

(51) Int. Cl. A61K 31/385; A61K 31/198; A61K 35/78

(52) U.S. Cl. 424/725; 514/440; 514/562; 514/561; 514/565

(57) ABSTRACT

The composition comprises (i) one or more ozonised oils and/or one or more synthetic and/or natural ozonised products, and (ii) thioctic acid and/or derivatives thereof, in which each one of the components is found in a concentration lying between 0.01% and 99.99% by weight with respect to the total, and, optionally, one or more active substances, additives, vehicles or excipients. This composition is suitable for the elaboration of pharmaceutical, cosmetic, dietary and food-supplement compositions in human beings and in animals.

### CROSS-REFERENCE TO RELATED APPLICATIONS

The present application claims priority from Spanish patent application No. 2,162,586, filed Nov. 25, 1999, and from, and is also a continuation-in-part application of, PCT patent application No. PCT/ES00/00208, filed Jun. 9, 2000, presently pending.

### FIELD OF THE INVENTION

The present invention relates to compositions that comprise ozonised oils and/or natural and/or synthetic ozonised products, and thioctic acid and/or derivatives thereof, along with, optionally, other additional components. Said compositions are suitable for the production of pharmaceutical, cosmetic and dietary and food supplement compositions, in human beings and animals.

### BACKGROUND OF THE INVENTION

Ozonised oils and natural or synthetic ozonised products are natural or synthetic products that contain molecules with carbon-carbon (olefinic) double bonds, which, on being submitted to a reaction with ozone, in suitable fashion, produce a mixture of ozonides, oligomeric ozonides and different products rich in active oxygen, of the peroxide, hydroperoxide type, etc. These substances are carriers of active oxygen and have a marked germicidal character, as well as possessing the capacity to stimulate different enzymatic processes of oxidation-reduction in cells, such as that of glutathion peroxidase, glutathion reductase, glucose-6-phosphatedehydrogenase, superoxide dismutase, catalase, etc., consuming reduction equivalents such as NADH and NADPH. The aforementioned processes have a direct implication in the revitalisation of many vital and/or cell specific functions. For more than 30 years, these products have been used in limited fashion, as a way of application of ozonotherapy, on the one hand by specialist in this field and in others, including the veterinary field. These products are of use in the local treatment in cases of infections, ulcers, crusts, burns and other tissue lesions of the gastrointestinal tract. Similarly, some attempts have been made in the field of cosmetics to treat acne and care for the skin, etc. There has also been observed evidence of systemic effects when taken orally, parenterally or in the blood. Many of these effects are attributed to its capacity to stimulate different oxidation-reduction enzymatic processes in cells, such as that of glutathion peroxidase, glutathion reductase, glucose-6-phosphatedehydrogenase, superoxide dismutase, catalase, etc., which have a direct implication in many vital and/or cell specific functions.

Thioctic acid, also known as alpha-lipoic acid, alpha-lipoate, 1.2-dithiolan-3-pentanoic acid, etc, presents properties such as that of a metabolic antioxidant. Thus, many enzyme systems in animals treat it as a substrate for bioreductions. Thioctic acid as a supplement is reduced to dihydrolipoate at the expense of cellular equivalents of reduction, such as NADH and NADPH. As the consumption of these reduction equivalents increases, the rate of cell metabolism also increases to make up for the increased demand. Thus, a property of thioctic acid is that of facilitating the use of the metabolic capacities of the cells themselves for recycling and enhancement. It is also considered capable of regenerating other natural antioxidants, such as vitamin C and E, glutathion, etc. Thioctic acid has been used in Europe for many years as an atoxic nutrient because of its capacity to help to maintain or restore hepatic health, as a treatment for several diseases related with toxins (mushroom poisoning, radiation, etc) and in alcoholic hepatitis. It has also been reported to be useful in patients with AIDS who receive antiviral or antibiotic treatments to contribute to the normalisation of the hepatic enzymes.

It has now been discovered, for the first time, that the association of ozonised oils and/or other natural and/or synthetic ozonised products with thioctic acid and/or derivatives thereof, along with, optionally, other substances of the type that will be mentioned later, acts as a perfect enhancer agent for the former, and said association is particularly advantageous in pharmaceutical, cosmetic, dietary and food supplement uses, in human and veterinary fields.

### DETAILED DESCRIPTION OF THE INVENTION

The invention provides a composition, hereinafter the composition of the invention, that comprises (i) one or more ozonised oils and/or one or more synthetic and/or natural ozonised products, and (ii) thioctic acid and/or derivatives thereof, in which each one of the components is found in a concentration lying between 0.01% and 99.99% by weight with respect to the total, preferably between 0.01% and 50% by weight.

The oils and natural or synthetic ozonised products can be obtained by means of the procedures described in the state of the art [Gabelein, K., “Therapeutische eigenschaften der aus ozongas gebildeten organische ozonide”, Erfahr. hk. 23, 167-173 (1974); Gabelein, K., “Eine ganz besondere qualitat der ozonverbindungen”, Erfahr. hk. 26, 203-209 (1977); German patent DE 12 55 660 (Gabelein, K., 1968); Japanese patent JP 55017351 (Rikagaku, K., 1980)].

In the sense used in this specification, the term “derivative” refers to substances constituted by the original molecule (in other words, the molecule from which they are derived), in which some of its constituent parts may be been substituted (chemical functional groups or atoms), by another chemical functional group, atom or molecule, in which the main structure is that of the original molecule. This term also includes the possibility that there has been performed the chemical addition of some other chemical functional group, atom or molecule to some of the bonds in the original structure.

The term “in any of its forms”, as used in this specification in relation to a chemical element, means any substance more complex, either organic or inorganic, complex, etc., that contains it.

Similarly, as used in this specification, the term “conjugate” means substances constituted by the original molecule, or an essential part thereof, chemically combined with another of approximately the same size, or bigger, generally of the type of bases, such as those that constitute DNA, or others related thereto or derived therefrom. Specifically applied to melatonin, the term “conjugate” makes reference to any more complex form that contains melatonin or derivatives thereof and which serves to transport them to specific structures of certain cells.

The composition of the invention can contain, if so desired, in variable quantities, one or more active substances and/or additives and/or vehicles and/or excipients suitable for pharmaceutical, cosmetic, dietary or as food additive use, in human or veterinary fields.

In a particular embodiment, the composition of the invention contains one or more active substances that significantly enhance their effectiveness, selected from among, for example, substances that facilitate the stimulation of cellular biochemical processes, such as oxidation-reduction of oxidising and/or reducing metabolites, glycolysis, oxidative decarboxylation of pyruvic acid, the Krebs cycle, the cellular respiratory chain, etc., as well as substances that facilitate synthesis at cellular level of glutathion, NAD-NADH, NADP-NADPH, flavonoids, enzymes, co-factors, etc. Among said active substances there are found, for illustrative purposes that do not limit the scope of the present invention, among others:

amino acids and derivatives thereof, for example, cysteine, arginine, glycine, N-acetyl-cysteine, etc., and derivatives thereof, at a concentration, in the event that they are present, lying between 0.01% and 25% by weight with respect to the total;

organic acids and derivatives thereof, for example, fumaric acid, succinic acid, eicosapentanoic acid, hyaluronic acid, pantotenic acid, etc., and derivatives thereof, in a concentration, in the event that they are present, lying between 0.01% and 25% by weight with respect to the total;

essential micro-elements and oligo-elements, for example, selenium, magnesium, manganese, zinc, copper, silver, etc., in any of their forms (salts, oxides, hydroxides, etc.), at a concentration, in the event that they are present, lying between 0.01% and 10% by weight with respect to the total;

vitamins, and precursors and derivatives thereof, for example, vitamins from groups A, B, C, D, E and F, their precursors and derivatives, at a concentration in the event that they were present, lying between 0.01% and 25% with respect to the total;

glutathion and its derivatives, acetylglucosamine and its derivatives, bioflavonoids and their derivates, coenzyme Q10 and its derivatives, melatonin and its derivatives and conjugates, mono-, sesqui-, di- or tri-terpenes and their derivatives, at a concentration in the event that they were present, lying between 0.01% and 10% with respect to the total;

essential fatty acids, for example, linoleic acids and acids of the Ω-3 and Ω-6 type, at a concentration in the event that they were present, lying between 0.01% and 50% with respect to the total;

phytoestrogens and their derivatives, alkaloids such as nicotine, johimbin, carnitine, galantoin, etc., their derivatives and precursors, at a concentration in the event that they were present, lying between 0.01% and 10% with respect to the total;

film-forming compounds, such as compounds that form protective films of the skin, for example, polyfluorocarbons, such as FOMBLIN (a polyfluorocarbon that forms a protective film on the skin and allows the exchange of oxygen) and similar, at a concentration in the event that they were present, lying between 0.01% and 10% with respect to the total; and

microorganisms and/or probiotic ferments such as lactobacillus LC1, rhamnosus GG, B. subtilis, etc., at a concentration in the event that they were present, lying between 1% and 20% with respect to the total.

The composition of the invention may contain one or more of the aforementioned active substances.

The composition of the invention is characterised by the association between (i) ozonised oils and/or other natural and/or synthetic ozonised products, and (ii) thioctic acid and/or derivatives thereof, optionally along with one or more of the active substances of the aforementioned type and in that said association presents an enhancing effect of the activities of said ozonised oils and natural or synthetic ozonised products, which is particularly advantageous in the uses of cosmetics, dietary products, as food supplements, and pharmaceutical products, for example, in the prevention and treatment of dermatitis, infections, acne, ulcers (including gastroduodenal ulcers), crusts, burns and other tissue lesions, including the gastrointestinal tract, various cellular dysfunctions, energy metabolism disorders and disorders of the enzymatic systems protecting against oxidants, ageing, effects derived from free radicals, conditions related with oxidative stress and stress in general, and others.

In the sense used in this description the term “oxidative stress” comprises any type of physiological and/or pathological condition related to an increase in the levels of peroxides and/or free radicals in general, whether due to abnormal reduction in the activity of the enzymes responsible for their metabolisation, and through this the control of said levels, or due to the coincidence of different factors that may condition its elevation, whether they be environmental, dietary, toxic, due to ageing, diseases, etc. Among the conditions that can be mentioned by way of example: physical or mental stress, fatigue, ageing, toxic habits, hepatic insufficiency, viral diseases, inflammatory and ischaemic processes, different types of dysmetabolisms, neurological diseases, etc. The increase in levels of free radicals and other oxidants, above the levels necessary for defence of the organism, produce damage due to oxidation in healthy tissues, especially when said situation extends in time, and at times, may lead to multi-organ failure.

The association of compounds provided by this invention also act on the metabolism of carbohydrates, fatty acids, lipids, in the cellular respiratory chain, etc., activating to a greater extent the mitocondrial functionality, as well as other functionalities.

A noticeable synergistic effect has been observed resulting in the combination of compounds provided by the present invention, which leads to much more intense effects of each one of the components.

It has also been found, in addition, that the compositions of the invention are useful for the preparation of medicaments, cosmetics, dietary compositions, food supplements, etc., for human and/or veterinary use.

Therefore, the invention also provides a pharmaceutical composition that comprises a composition of the invention and one or more pharmaceutically acceptable excipients and/or vehicles. In a particular embodiment, the invention provides a pharmaceutical composition suitable for the prevention and treatment of cellular dysfunctions related to oxidative stress and to the poor functioning of cellular biochemical processes in general.

The cosmetic compositions provided by this invention comprise a composition of the invention along with one or more suitable vehicles and/or excipients.

Similarly, the dietary compositions and the nutritional supplements provided by this invention comprise a composition of the invention along with one or more additives and/or vehicles, matrices or acceptable supports.

The following examples serve to illustrate the present invention and should not be considered as limiting the scope thereof.

### EXAMPLE 1

Obtaining Ozonised Olive Oil

Ozonised olive oil can be obtained by directly ozonising food-grade olive oil with ozone at room temperature.

1 kg of olive oil is placed in a sterile vertical glass column reactor, which possesses, in its interior, an entrance for gas via a sintered porous glass disk. Next, a flow of 3 l/h (liters/hour) of a mixture of ozone and oxygen from an ozone generator with approximately 60 mg of ozone/l is made to pass through sintered glass disk. In order to obtain the ozone, medicinal oxygen is used, which, by means of a suitable reduction in pressure, feeds an ozone generator using silent electrical discharge. By controlling the voltage of the feed to the ozone generator and the flow of gas, the desired concentration of ozone in the gaseous mixture can be established.

Ozonisation is performed continually for several hours until it is observed that the mixture from the reactor begins to solidify. At this time, the ozonisation is detained and the ozonised oil is extracted. The product obtained is washed with a saturated solution of sodium bicarbonate in bi-distilled water and dried with anhydrous sodium sulphate. Next, the acidity index of the product is determined, which should not be greater than 15. If it is greater than this value, the product is washed once more with a solution of sodium bicarbonate until reaching the desired acidity. When this has been achieved, the product is bottled in a sterile container, covered and labelled.

The final product obtained contains part of the initial unreacted olive oil, and a mixture of monomeric and oligomeric ozonides, peroxides, hydroperoxides, etc., all of them rich in active oxygen.

### EXAMPLE 2

Capsules for Gastroduodenal Ulcers

Capsules

Some capsules are prepared with the following composition:

| Component | Percentage by weight (%) |
|---|---|
| Micronised activated silica gel | 50 |
| Ozonised sunflower oil | 48 |
| Thioctic acid | 1.9 |
| Melatonin | 0.1 |

960 g of ozonised oil are heated to 40° C. in a hot-water bath, and in this oil, 38 g of thioctic acid and 2 g of melatonin are dissolved. After this, the mixture is cooled to room temperature. Next, 1 kg of micronised activated silica gel is introduced into a high-speed mixer. This is then covered and the mixture agitated. Little by little, the previous mixture is added through a side entrance port, until there is total absorption thereof. The product is encapsulated in gastric capsules (of gelatine), these containing 1 g of product.

Efficacy Assay

The efficacy of the encapsulated product was evaluated in 10 patients with ages lying between 29 and 61 years, who had gastroduodenal ulcers of sizes lying between 0.5 and 2 cm diameter, and whose gastric cultures were positive for the presence of Helicobacter pylori.

The indicated treatment consisted of taking a capsule of the product on getting up, before eating, and another on going to bed, more than 2 hours after the last meal of the day, for the first 7 days, and taking 1 capsule of the product on going to bed, more than 2 hours after the last meal of the day, for 35 days.

An evaluation was performed after 21 days and another at the end of the treatment. The results obtained are shown in Table 1.

TABLE 1
Results of the efficacy assay (capsules)

| | |
|---|---|
| Examination | Examination |
| after 21 days | after 42 days |

| Patient | Age | Ulcer | H. pylori | Ulcer | H. pylori |
|---|---|---|---|---|---|
| FR | 35 | + | − | − | − |
| AG | 56 | + | − | − | − |
| MB | 61 | − | − | − | − |
| RA | 59 | + | − | − | − |
| PD | 42 | + | + | − | − |
| GP | 37 | + | − | − | − |
| CI | 49 | + | − | − | − |
| MF | 29 | + | + | + | + |
| GT | 52 | + | − | − | − |
| MR | 47 | + | − | − | − |

Nine patients showed healing of the ulcers (−) and cultures negative (−) to the presence of H. pylori at the end of the treatment. Only one patient remained positive for H. pylori (+) and a gastric ulcer (+) present at the end of the treatment, although it is worth pointing out that their size had reduced by 50%. No complications or side effects were observed during the treatment.

### EXAMPLE 3

Post-resurfacing Epithelial Repairing Cream

Cream

A post-resurfacing epithelial repairing cream is prepared with the following composition:

| Component | Percentage by weight (%) |
|---|---|
| 1 Ozonised jojoba oil L15 | 10 |
| 2 Thioctic acid | 1 |
| 3 Glutathion | 0.1 |
| 4 Colloidal silver | 3 |
| 5 Acemulgor LAM | 9 |
| 6 Cetyl alcohol | 1.5 |
| 7 Stearin | 1.5 |
| 8 Cetiol 868 | 1 |
| 9 Cetiol V | 2 |
| 10 Isopropyl stearate | 0.2 |
| 11 DC 345 | 2 |
| 12 Avocado oil | 1 |
| 13 Panalane | 3 |
| 14 Jojoba oil | 2 |
| 15 DC 200-350 | 2 |
| 16 Carbopol Ultrez | 0.5 |
| 17 Deionised water | 57 |
| 18 18 beta-glycolic acid | 0.5 |
| 19 Vitamin E | 0.1 |
| 20 Liquapar PE | 0.8 |
| 21 EDTA Na2 | 0.1 |
| 22 Glycerine | 1.5 |
| 23 Controx KS | 0.1 |
| 24 Perfume Dersit | 0.1 |

The product is prepared as a cosmetic cream, mixing the components in a turboemulsifier. The different components (from the fourth to the 22^{nd}) are mixed with the usual procedure. The ozonised L15 jojoba oil is heated to 40° C. in a hot-water bath, and the thioctic acid and glutathion are dissolved therein, cooling straight away. This mixture is added to the remaining components in the turboemulsor after the different “oil” phases. Once the emulsion has formed, this is rapidly cooled. Once cool, the cream is packaged in 50-ml plastic tubes.

Efficacy Assay

The efficacy of the product was evaluated in 10 subjects with ages ranging from 43 to 64 years. These subjects had been submitted to a laser facial resurfacing treatment. The cream was applied immediately after treatment and, two times a day for the following days.

The effects following resurfacing treatments are usually stinging, scab formations, reddening and inflammation. The attenuate these sequelae, moisturising cosmetic creams are used with anti-oxidant components, but, often, these sequelae last for 7 or 8 days and, in some cases, are complicated with extra infections.

The evolution of sequelae was evaluated for the group of subjects studied by means of an evaluation scale of degree of intensity, ranging from 1 to 3+, at 24 hours and 3 days after treatment.

1. The results obtained are presented in Table 2.

TABLE 2
Results of the efficacy assay (cream)

| | | 24 h | | | | 3 days | | | |
|---|---|---|---|---|---|---|---|---|---|
| Subject | Age | St | Sc | Red | Inf | St | Sc | Red | Inf |
| AV | 46 | ++ | ++ | +++ | ++ | − | + | + | − |
| RM | 58 | + | ++ | ++ | ++ | − | − | − | − |
| GL | 43 | + | + | ++ | ++ | − | − | − | − |
| GP | 64 | ++ | ++ | +++ | +++ | − | − | + | − |
| RL | 56 | | ++ | ++ | ++ | − | − | − | − |
| CP | 46 | ++ | +++ | +++ | +++ | − | − | − | − |
| AU | 49 | + | ++ | + | + | − | − | − | − |
| MC | 47 | +++ | ++ | ++ | ++ | − | + | + | + |
| SO | 63 | ++ | + | + | ++ | − | − | − | − |
| AO | 52 | +++ | | +++ | ++ | − | − | − | − |

[St: Stinging; Sc: Scabs; Red: Reddening; Inf: Inflammation]

The results showed that the sequelae resurfacing treatment were, in general, less severe, and that the evolution towards their disappearance was quicker than with the use of other creams. After 3 days, no subject had stinging, only two maintained a small scab, 3 had slight reddening, and only one presented very slight inflammation. In no case was there any infection.

### EXAMPLE 4

Yoghurt Enriched with Ozonised Vegetal Oil and Thioctic Acid for the Regulation of Slow and Incomplete Digestion and Intestinal Upsets

Yoghurts were prepared with ozonised vegetal oil and thioctic acid, suitable for the regulation of slow and incomplete digestions and intestinal upsets. They had the following composition:

| Component | Percentage by weight (%) |
|---|---|
| Yoghurt, unflavoured and no added sugar | 99.5 |
| Ozonised L15T15 sunflower oil | 0.2 |
| Thioctic acid | 0.1 |
| Vitamin C | 0.1 |
| Glycine | 0.1 |

For the preparation of the yoghurt, to each 995 g of yoghurt there are added in sterile conditions 2 g of ozonised sunflower oil, 1 g of thioctic acid, 1 g of vitamin C and 1 g of glycine, mixing all in cold conditions. The mixture is cooled, and packaged in proportions of 100 g and kept refrigerated (1-10° C.).

Efficacy Assay

The efficacy of the product in a group of subjects suffering frequently from slow and heavy digestion, as well as frequent gastrointestinal upsets, accompanied by episodes of diarrhoea was evaluated.

The subjects were recommended to take 100 g of prepared yoghurt with not sugar after each meal for 35 days.

The frequency of slow and heavy digestions, as well as episodes of diarrhoea was evaluated with four levels: frequent (+++), not very frequent (++), few (+) and none (−), 21 days after beginning the study and at the end thereof. The results obtained are shown in Table 3.

TABLE 3
Results of the efficacy assay (yoghurt)

| | | Examination | | Examination | |
|---|---|---|---|---|---|
| | | 21 days after | | 35 days after | |
| | | Slow | | Slow | |
| Patient | Age | digestion | Diarrhoea | digestion | Diarrhoea |
| PT | 35 | ++ | + | − | − |
| AM | 56 | ++ | ++ | − | − |
| EL | 61 | +++ | +++ | + | + |
| DT | 59 | ++ | + | − | − |
| AF | 42 | + | ++ | − | − |
| MP | 37 | + | + | − | − |
| NJ | 49 | +++ | ++ | + | − |
| VB | 29 | ++ | + | − | − |
| FC | 52 | + | + | − | − |
| BR | 47 | ++ | ++ | − | − |

As can be seen, the product was effective, as most of the subjects resolved their gastrointestinal problems. Only 2 subjects maintained infrequent episodes of heavy digestion, and one of them, infrequent episodes of occasional diarrhoea.

### EXAMPLE 5

Capsules of ozonised vegetal oil and thioctic acid for increasing the vitality and control of fat and weight

Capsules

Capsules are prepared with the following composition:

| Component | Percentage by weight (%) |
|---|---|
| Micronised clay | 80 |
| Ozonised sunflower oil L15T15 | 16.5 |
| Thioctic acid | 0.4 |
| Lactobacilli LC1 | 3 |
| Selenium methionine | 0.1 |

165 g of ozonised oil are heated to 40° C. in a hot-water bath, and the following ingredients mixed in the following order: 4 g of thioctic acid and 1 g of selenium methionine. Next, the mixture is cooled to room temperature and 30 g of lactobacilli LC1 added, mixing until obtaining a homogeneous dispersion. Next, 800 g of micronised clay are introduced in a high-speed mixer, the mixer covered and agitation thereof started. Little by little, the previous mixture is added through a side orifice until complete absorption of the mixture is attained. The product is encapsulated in gastric capsules (of gelatine), with 1 g of product.

Efficacy Assay

The efficacy of the product was evaluated in 20 subjects with ages ranging from 25 to 57 years, who showed frequent symptoms of physical exhaustion and who presented various degrees of overweight.

The following treatment was indicated: perform 6 hours of moderate physical exercise a week (fitness), observe a maintenance diet of 3 kcal, take a capsule of the product on getting up before eating anything and another on going to bed, at least 2 hours after the last food of the day, for the first 10 days, and 1 capsule of product on going to bed, at least 2 hours after the last food of the day, for the other 32 days.

An evaluation was performed before starting treatment, another after 21 days and another at the end of treatment, in which the weight and percentage fat was measured by plicometry, and the sensation of physical exhaustion during the morning, evening and night, in degrees of intensity 1 and 3+. The results obtained are presented in Table 4.

As can be observed, all subjects lost weight, and most notably, the amount of fat. According to the means, a subject with a weight of 79 kg at the beginning and 18.2 kg of fat (79×0.23) loses, in the study period, 3.2 kg of total weight and loses 4.2 kg of fat; the difference can represent an increase in muscle mass. Regarding the physical exhaustion, this disappeared in most of the subjects and, in those in which it was still manifest, was of much lower intensity and frequency.

TABLE 4
Results of the efficacy assay (capsules)

| | | | Initial Exam | | | | | Exam After 21 Days | | | | | Exam After 42 Days | | | | |
|---|---|---|---|---|---|---|---|---|---|---|---|---|---|---|---|---|---|
| | | | Physical exhaustion | | | | | Physical exhaustion | | | | | Physical exhaustion | | | | |
| | | | | | | Weight | | | | | Weight | | | | | Weight | |
| SUB | SEX | AGE | M | E | N | (kg) | % Fat | M | E | N | (kg) | % Fat | M | E | N | (kg) | % Fat |
| CM | M | 26 | X | X | XXX | 78.4 | 22 | 0 | X | X | 77.2 | 20 | 0 | 0 | 0 | 75.3 | 17 |
| AR | M | 42 | XXX | X | X | 84.6 | 26 | X | 0 | X | 82.7 | 22 | 0 | 0 | 0 | 81.4 | 20 |
| RB | F | 45 | XX | X | XX | 66.5 | 20 | X | 0 | 0 | 66.0 | 19 | 0 | 0 | 0 | 66.2 | 18 |
| GA | M | 35 | 0 | XX | XXX | 81.7 | 32 | 0 | X | X | 78.5 | 27 | 0 | 0 | 0 | 77.4 | 24 |
| PM | M | 38 | X | XXX | XX | 76.3 | 28 | 0 | X | X | 75.1 | 25 | 0 | 0 | 0 | 73.4 | 22 |
| AA | F | 29 | XX | X | XXX | 67.4 | 18 | X | 0 | XX | 66.1 | 17 | 0 | 0 | X | 66.7 | 16 |
| SN | F | 27 | 0 | XX | XXX | 69.7 | 17 | 0 | X | X | 69.5 | 17 | 0 | 0 | 0 | 68.0 | 15 |
| PS | F | 25 | XXX | 0 | X | 64.2 | 18 | X | 0 | X | 65.7 | 16 | 0 | 0 | 0 | 64.8 | 13 |
| RA | M | 38 | XX | 0 | XX | 94.5 | 24 | X | 0 | X | 92.3 | 21 | 0 | 0 | 0 | 89.3 | 18 |
| ET | M | 43 | 0 | X | XXX | 88.5 | 18 | 0 | 0 | X | 87.5 | 16 | 0 | 0 | 0 | 85.4 | 14 |
| CM | M | 41 | X | XX | 0 | 82.7 | 20 | 0 | X | 0 | 81.7 | 17 | 0 | 0 | 0 | 78.4 | 13 |
| FU | M | 57 | XXX | X | XXX | 99.4 | 26 | X | 0 | XX | 97.2 | 23 | 0 | 0 | X | 92.6 | 18 |
| MR | F | 27 | XX | 0 | X | 64.3 | 18 | X | 0 | 0 | 65.2 | 18 | X | 0 | 0 | 65.0 | 17 |
| RM | F | 45 | XXX | 0 | X | 56.9 | 16 | X | 0 | 0 | 57.3 | 16 | 0 | 0 | 0 | 57.6 | 16 |
| NB | F | 32 | 0 | XXX | 0 | 75.6 | 18 | 0 | X | 0 | 75.1 | 17 | 0 | X | 0 | 74.2 | 15 |
| TG | F | 33 | X | X | X | 69.8 | 17 | 0 | 0 | 0 | 69.3 | 16 | 0 | 0 | 0 | 68.3 | 15 |
| GB | M | 43 | 0 | X | XX | 87.2 | 23 | 0 | 0 | 0 | 84.6 | 19 | 0 | 0 | 0 | 81.3 | 14 |
| AZ | M | 36 | 0 | XXX | XXX | 104.3 | 32 | 0 | XX | XX | 99.8 | 27 | 0 | 0 | X | 94.9 | 22 |
| IC | F | 39 | XX | X | 0 | 77.8 | 28 | X | 0 | 0 | 76.3 | 25 | 0 | 0 | 0 | 74.1 | 22 |
| BO | F | 44 | XXX | XX | X | 89.7 | 39 | X | X | X | 85.6 | 33 | 0 | 0 | 0 | 81.8 | 28 |
| | Me | | | | | 79.0 | 23.0 | | | | 77.6 | 20.6 | | | | 75.8 | 17.9 |

[SUB: Subject; M: Morning; E: Evening; N: Night; Me: Mean]

1. A composition that comprises (i) at least one of ozonised oils, natural and synthetic ozonised products, and (ii) at least one of thioctic acid and derivatives thereof, in which each one of the components is found at a concentration lying between 0.01% and 99.99% by weight with respect to the total.

2. A composition according to claim 1, in which each one of the components is found at a concentration lying between 0.01% and 50% by weight with respect to the total.

3. A composition according to claim 1, which comprises, in addition, at least one of suitable active substances, additives, vehicles and excipients for pharmaceutical, cosmetic, dietary, or food supplement use, in the human and veterinary fields.

4. A composition according to claim 3, which comprises, in addition, an active substance selected from an amino acid, a derivative of an amino acid and mixtures thereof, at a concentration lying between 0.01% and 25% by weight with respect to the total.

5. A composition according to claim 4, in which said active substance is selected from cysteine, arginine, glycine, N-acetyl-cysteine, derivatives thereof and mixtures thereof, at a concentration lying between 0.01% and 25% by weight with respect to the total.

6. A composition according to claim 3, which comprises, in addition, an active substance selected from an organic acid, a derivative of an organic acid and mixtures thereof, at a concentration lying between 0.01% and 25% by weight with respect to the total.

7. A composition according to claim 6, in which said active substance is selected from fumaric acid, succinic acid, eicosapentanoic acid, hialuronic acid, pentatonic acid, derivatives thereof, and mixtures thereof, at a concentration lying between 0.01% and 25% by weight with respect to the total.

8. A composition according to claim 3, which comprises, in addition, an active substance selected from vitamins, precursors thereof, derivatives thereof and mixtures thereof, at a concentration lying between 0.01% and 25% by weight with respect to the total.

9. A composition according to claim 8, in which said active substance is selected from a vitamin of groups A, B, C, D, E, F, their precursors, derivatives thereof and mixtures thereof, at a concentration lying between 0.01% and 25% by weight with respect to the total.

10. A composition according to claim 3, which comprises, in addition, at least one of an active substance selected from glutathion and derivatives thereof, acetylglucosamine and derivatives thereof, bioflavonoids and derivatives thereof, coenzyme Q10 and derivatives thereof, and mixtures thereof, at a concentration lying between 0.01% and 10% by weight with respect to the total.

11. A composition according to claim 3, which comprises, in addition, at least one of an active substance selected from melatonin and derivatives thereof, and mono-, sesqui-, di- or tri-terpenes and derivatives thereof, at a concentration lying between 0.01% and 10% by weight with respect to the total.

12. A composition according to claim 3, which comprises, in addition, at least, an active substance selected from essential fatty acids and/or derivatives thereof, at a concentration lying between 0.01% and 50% by weight with respect to the total.

13. A composition according to claim 12, in which said active substance is selected from linoleic acid and acids of the type Ω-3, Ω-6, and mixtures thereof, at a concentration lying between 0.01% and 50% by weight with respect to the total.

14. A composition according to claim 3, which comprises, in addition, at least one of an active substance selected from phytoestrogens and derivatives thereof, and alkaloids, derivatives and precursors thereof, at a concentration lying between 0.01% and 10% by weight with respect to the total.

15. A composition according to claim 14, in which said active substance is selected from nicotine, yohimbin, carnitine, galantoin, derivatives and precursors thereof, and mixtures thereof, at a concentration lying between 0.01% and 10% by weight with respect to the total.

16. A composition according to claim 3, which comprises, in addition, at least an active substance selected from film-forming compounds, at a concentration lying between 0.01% and 10% by weight with respect to the total.

17. A composition according to claim 16, in which said active substance is selected from polyfluorocarbons forming films protecting the skin, at a concentration lying between 0.01% and 10% by weight with respect to the total.

18. A composition according to claim 3, which comprises, in addition, at least one of an active substance selected from micro-organisms and probiotic ferments, at a concentration lying between 1% and 20% by weight with respect to the total.

19. A composition according to claim 18, in which said active substance is selected from lactobacillus LC1, rhamnosus GG, B. subtilis, and mixtures thereof, at a concentration lying between 1% and 20% by weight with respect to the total.

20. A pharmaceutical composition that comprises a composition according to claim 3 and at least one of pharmaceutically acceptable excipients and vehicles.

21. A cosmetic composition that comprises a composition according to claim 3 and at least one of acceptable excipients and vehicles.

22. A dietary composition that comprises a composition according to claim 3 and at least one of acceptable additives and vehicles.

23. A food supplement that comprises a composition according to claim 3 and at least one of acceptable additives and vehicles.

24. A composition according to claim 20 for the prevention and treatment of dermatitis, infections, acne, ulcers, including gastroduodenal ulcers, crusts, burns, tissue lesions, various cellular dysfunctions, energy metabolism disorders and disorders of enzymatic systems protecting against oxidants, ageing, effects derived from free radicals, conditions related to oxidative stress and to stress in general in humans and/or in animals.

25. A composition according to at least one of claim 22 and 23 for the improvement of at least one of nutrition and health in at least one of humans and animals.

26. A cosmetic composition for at least one of cosmetic personal care and cosmetic treatment that comprises a composition according to claim 3 and at least one of cosmetically acceptable excipients and vehicles.

27. A method of making a medicament comprising use of a composition according to claim 3 for the preparation of a medicament for the prevention and treatment of dermatitis, infections, acne, ulcers, including gastroduodenal ulcers, crusts, burns, tissue lesions, various cellular dysfunctions, energy metabolism disorders and disorders of enzymatic systems protecting against oxidants, ageing, effects derived from free radicals, conditions related to at least one of oxidative stress and stress in general in at least one of humans and in animals.

28. A method of making a cosmetic composition comprising use of a composition according to claim 3 for the preparation of a cosmetic for at least one of personal care and cosmetic treatment for such treatment in at least one of humans and animals.

29. A method of making at least one of a dietary composition and a food supplement comprising use of a composition according to claim 3 for the preparation of a dietary composition for the improvement of at least one of human nutrition, human health, nutrition treatment in at least one of humans and animals, and health treatment in at least one of humans and animals.

30. A method of using a cosmetic composition comprising use of the composition according to claim 3 as a cosmetic for at least one of personal care, cosmetic treatments and such treatments in at least one of humans and animals.
